(19) United States
(12) Patent Application Publication
(10) Pub. No.: US 2026/0308089 A1
(43) Pub. Date: Oct. 8, 2026

(54) AZEOTROPIC COMPOSITIONS COMPRISING HYDROGEN FLUORIDE

(71) Applicant: THE CHEMOURS COMPANY FC, LLC, WILMINGTON, DE (US)

(72) Inventors: SHENG PENG, HOCKESSIN, DE (US); SIDDARTH SITAMRAJU, MANVEL, TX (US); OLAGAPPAN MUTHU, NEWARK, DE (US)

(73) Assignee: THE CHEMOURS COMPANY FC, LLC, WILMINGTON, DE (US)

(21) Appl. No.: 19/483,058

(22) PCT Filed: Jun. 7, 2024
(86) PCT No.: PCT/US2024/032897

§ 371 Date: Nov. 10, 2025

Related U.S. Application Data
(60) Provisional application No. 63/471,842, filed on Jun. 8, 2023.

(51) Int. Cl. C07C 17/386; B01D 3/36

(52) U.S. Cl. CPC C07C 17/386; B01D 3/36

(57) ABSTRACT

The present application discloses azeotrope and azeotrope-like compositions comprising hydrogen fluoride and a compound selected from 2,4-dichloro-1,1,1,4,4-pentafluorobutane (345lfd) and 3-chloro-1,1,4,4,4-pentafluorobut-1-ene (1335czd).

### FIELD

This invention relates to azeotrope and azeotrope-like compositions comprising hydrogen fluoride and a compound selected from 2,4-dichloro-1,1,1,4,4-pentafluorobutane (345lfd) and 3-chloro-1,1,4,4,4-pentafluorobut-1-ene (1335czd).

### BACKGROUND

Many industries have been working for the past few decades to find replacements for the ozone depleting chlorofluorocarbons (CFCs) and hydrochlorofluorocarbons (HCFCs). The CFCs and HCFCs have been employed in a wide range of applications, including their use as aerosol propellants, refrigerants, cleaning agents, expansion agents for thermoplastic and thermoset foams, heat transfer media, gaseous dielectrics, fire extinguishing and suppression agents, power cycle working fluids, polymerization media, particulate removal fluids, carrier fluids, buffing abrasive agents, and displacement drying agents. In the search for replacements for these versatile compounds, many industries have turned to the use of hydrofluorocarbons (HFCs) and hydrofluoroolefins (HFOs).

### SUMMARY

The present application provides, inter alia, compositions comprising: i) hydrogen fluoride; and ii) a compound selected from 2,4-dichloro-1,1,1,4,4-pentafluorobutane (345lfd) and 3-chloro-1,1,4,4,4-pentafluorobut-1-ene (1335czd); wherein the 2,4-dichloro-1,1,1,4,4-pentafluorobutane (345lfd) or 3-chloro-1,1,4,4,4-pentafluorobut-1-ene (1335czd) is present in the composition in an amount effective to form an azeotrope composition or azeotrope-like composition with the hydrogen fluoride.

Unless otherwise defined, all technical and scientific terms used herein have the same meaning as commonly understood by one of ordinary skill in the art to which this invention belongs. Methods and materials are described herein for use in the present invention; other, suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control.

### BRIEF DESCRIPTION OF THE DRAWINGS

FIG. 1 shows an exemplary vapor-liquid equilibrium plot representative of the properties of heterogeneous azeotropes.

### DETAILED DESCRIPTION

New environmental regulations on refrigerants have forced the refrigeration and air-conditioning industry to look for new refrigerants with low global warming potential (GWP). Replacement refrigerants are being sought that have low GWP, low toxicity, low or no flammability, reasonable cost and excellent refrigeration performance. Fluoroolefins satisfy these criteria.

HFCs do not contribute to the destruction of stratospheric ozone, but are of concern due to their contribution to the “greenhouse effect”, i.e., they contribute to global warming. As a result of their contribution to global warming, HFCs have come under scrutiny, and their widespread use may also be limited in the future. Thus, there is a need for compositions that do not contribute to the destruction of stratospheric ozone and also have low global warming potentials (GWPs). The formation of azeotropic or azeotrope-like compositions comprising hydrogen fluoride and a compound described herein (e.g., 2,4-dichloro-1,1,1,4,4-pentafluorobutane (345lfd) or 3-chloro-1,1,4,4,4-pentafluorobut-1-ene (1335czd)) allow said compounds to be removed more easily and at lower temperatures from other higher boiling by-products formed, for example, in the synthesis of (E)-1,1,1,4,4,4-hexafluoro-2-butene, allowing for improved purification of the desired product at lower energy and reduced cost. Additionally, said azeotropic compositions can be used in azeotropic distillations to remove hydrogen fluoride from higher boiling compounds that also form azeotropes with hydrogen fluoride. The azeotropic compositions described herein are minimum boiling azeotropes, that is, at a given pressure, the boiling temperature of the azeotrope is less than the boiling temperature of each of the individual components. These compositions may therefore be useful for isolating the azeotropic mixture at a lower temperature compared to the individual components.

Exemplary processes for preparing (E)-1,1,1,4,4,4-hexafluoro-2-butene may be found, for example, in U.S. Pat. Nos. 10,611,709 and 11,390,574, the disclosures of each of which are incorporated herein by reference in their entireties.

### Definitions and Abbreviations

As used herein, the terms “comprises,” “comprising,” “includes,” “including,” “has,” “having” or any other variation thereof, are intended to cover a non-exclusive inclusion. For example, a process, method, article, or apparatus that comprises a list of elements is not necessarily limited to only those elements but may include other elements not expressly listed or inherent to such process, method, article, or apparatus. Further, unless expressly stated to the contrary, “or” refers to an inclusive or and not to an exclusive or. For example, a condition A or B is satisfied by any one of the following: A is true (or present) and B is false (or not present), A is false (or not present) and B is true (or present), and both A and B are true (or present).

Also, use of “a” or “an” are employed to describe elements and components described herein. This is done merely for convenience and to give a general sense of the scope of the invention. This description should be read to include one or at least one and the singular also includes the plural unless it is obvious that it is meant otherwise.

As used herein, the term “about” is meant to account for variations due to experimental error (e.g., plus or minus approximately 10% of the indicated value). All measurements reported herein are understood to be modified by the term “about”, whether or not the term is explicitly used, unless explicitly stated otherwise.

When an amount, concentration, or other value or parameter is given as either a range, preferred range or a list of upper preferable values and/or lower preferable values, this is to be understood as specifically disclosing all ranges formed from any pair of any upper range limit or preferred value and any lower range limit or preferred value, regardless of whether ranges are separately disclosed. Where a range of numerical values is recited herein, unless otherwise stated, the range is intended to include the endpoints thereof, and all integers and fractions within the range.

Global warming potential (GWP) is an index for estimating relative global warming contribution due to atmospheric emission of a kilogram of a particular greenhouse gas compared to emission of a kilogram of carbon dioxide. GWP can be calculated for different time horizons showing the effect of atmospheric lifetime for a given gas. The GWP for the 100-year time horizon is commonly the value referenced.

As recognized in the art, an azeotropic composition is an admixture of two or more different components which, when in liquid form and (1a) under a given constant pressure, will boil at a substantially constant temperature, which temperature may be higher or lower than the boiling temperatures of the individual components, or (1b) at a given constant temperature, will boil at a substantially constant pressure, which pressure may be higher or lower than the boiling pressure of the individual components, and (2) will boil at substantially constant composition, which phase compositions, while constant, are not necessarily equal. (See e.g., M. F. Doherty and M. F. Malone, Conceptual Design of Distillation Systems, McGraw-Hill (New York), 2001, 185).

A homogeneous azeotrope, in which a single vapor phase is in equilibrium with a single liquid phase, has, in addition to properties (1a), (1b), and (2) above, the property that the composition of each component is the same in each of the coexisting equilibrium phases. The general term “azeotrope” is a commonly used alternative name for a homogeneous azeotrope.

A heterogeneous azeotrope, in which a single vapor phase is in equilibrium with two liquid phases, has properties (1a), (1b), and (2) as described above where, while constant, the three coexisting equilibrium phases each have different compositions (See e.g., M. F. Doherty and M. F. Malone, Conceptual Design of Distillation Systems, McGraw-Hill (New York), 2001, 352). At the heterogeneous azeotrope, the composition of the overall liquid phase, (i.e., the liquid phase composition obtained by combining the two equilibrium liquid phases), is identical to the composition of the equilibrium vapor phase.

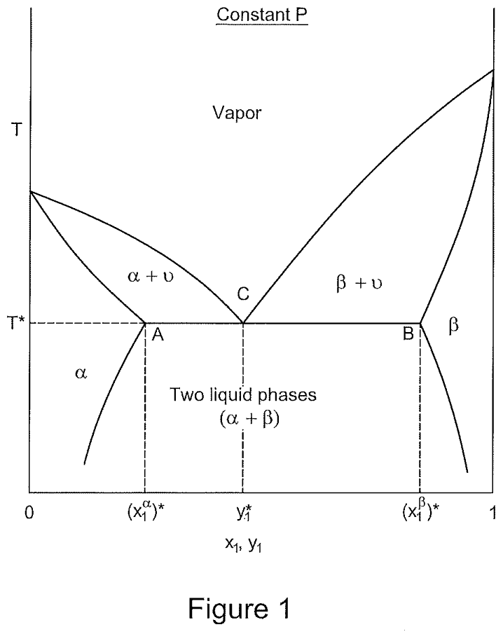

The properties of heterogeneous azeotropes are described, for example, in FIG. 1. As shown in FIG. 1, temperature T* is the minimum temperature at which both vapor and liquid phases occur. Below T*, depending on composition, liquid phase a, liquid phase, or both a and B will exist. When the liquid composition lies within the (α+β) region, it splits into two separate liquid phases whose compositions are defined by the intersection of the solid lines starting at points A and B and continuing toward the bottom of FIG. 1 and a horizontal line at the specified temperature (FIG. 1). Above T*, in regions (α+ν) and (β+ν), a single liquid phase is in equilibrium with a vapor phase whose compositions are indicated by the intersection of the solid lines bounding region (α+ν) or (β+ν), depending on the mixture composition, and a horizontal line at the specified temperature. At temperature T*, three phases of different compositions, denoted by A, B, and C in FIG. 1, are in equilibrium with each other. Any mixture with a liquid phase composition between (x₁^{α})* and (x₁^{β})* as shown in FIG. 1 will (I) separate into two liquid phases with compositions equal to (x₁^{α})* and (x₁^{β})*, (II) be in equilibrium with a vapor phase of composition y₁*, and (III) boil at the same temperature T*. Points A, B, and C and temperature T* of FIG. 1 constitute the three equilibrium phases of a heterogeneous azeotrope. At the azeotropic composition, the overall liquid phase composition equals y₁*.

As used herein, an “azeotrope-like” composition refers to a composition that behaves like an azeotropic composition (i.e., has constant boiling characteristics or a tendency not to fractionate upon boiling or evaporation). Hence, during boiling or evaporation, the vapor and liquid compositions, if they change at all, change only to a minimal or negligible extent. In contrast, the vapor and liquid compositions of non-azeotrope-like compositions change to a substantial degree during boiling or evaporation.

As used herein, the terms “azeotrope-like” or “azeotrope-like behavior” refer to compositions that exhibit dew point pressure and bubble point pressure with virtually no pressure differential. In some embodiments, the difference in the dew point pressure and bubble point pressure at a given temperature is 3% or less. In some embodiments, the difference in the bubble point and dew point pressures is 5% or less.

The term “compound” as used herein is meant to include all stereoisomers, geometric isomers, tautomers, and isotopes of the structures depicted. Compounds herein identified by name or structure as one particular tautomeric form are intended to include other tautomeric forms unless otherwise specified.

### Chemicals, Abbreviations, and Acronyms

1335czd: 3-chloro-1,1,4,4,4-pentafluorobut-1-ene
345lfd: 2,4-dichloro-1,1,1,4,4-pentafluorobutane
NRTL: Non-Random, Two-Liquid
psia: pounds per square inch absolute
VLE: vapor-liquid equilibrium

### Compositions

The present application provides compositions comprising: i) hydrogen fluoride; and ii) a compound selected from 2,4-dichloro-1,1,1,4,4-pentafluorobutane (345lfd) and 3-chloro-1,1,4,4,4-pentafluorobut-1-ene (1335czd); wherein the 2,4-dichloro-1,1,1,4,4-pentafluorobutane (345lfd) or 3-chloro-1,1,4,4,4-pentafluorobut-1-ene (1335czd) is present in the composition in an amount effective to form an azeotrope composition or azeotrope-like composition with the hydrogen fluoride.

In some embodiments, the composition provided herein comprises hydrogen fluoride and 2,4-dichloro-1,1,1,4,4-pentafluorobutane (345lfd), wherein the 2,4-dichloro-1,1,1,4,4-pentafluorobutane (345lfd) is present in the composition in an amount effective to form an azeotrope composition or azeotrope-like composition with the hydrogen fluoride.

In some embodiments, the composition comprises from about 83 to about 99.99 mole percent hydrogen fluoride, for example, from about 83 to about 99, about 83 to about 95, about 83 to about 90, about 83 to about 85, about 85 to about 99.99, about 85 to about 99, about 85 to about 95, about 85 to about 90, about 90 to about 99.99, about 90 to about 99, about 90 to about 95, about 95 to about 99.99, about 95 to about 99, or about 99 to about 99.99 mole percent hydrogen fluoride.

In some embodiments, the composition comprises about 0.01 to about 17 mole percent 2,4-dichloro-1,1,1,4,4-pentafluorobutane (345lfd), for example, from about 0.01 to about 15, about 0.01 to about 10, about 0.01 to about 5, about 0.01 to about 1, about 1 to about 17, about 1 to about 15, about 1 to about 10, about 1 to about 5, about 5 to about 17, about 5 to about 15, about 5 to about 10, about 10 to about 17, about 10 to about 15, or about 15 to about 17 mole percent 2,4-dichloro-1,1,1,4,4-pentafluorobutane (345lfd).

In some embodiments, the composition comprises from about 83 to about 99.99 mole percent hydrogen fluoride and from about 0.01 to about 17 mole percent 2,4-dichloro-1,1,1,4,4-pentafluorobutane (345lfd).

In some embodiments, the composition comprises from about 88.7 to about 99.99 mole percent hydrogen fluoride and from about 0.01 to about 11.3 mole percent 2,4-dichloro-1,1,1,4,4-pentafluorobutane (345lfd).

In some embodiments, the composition comprising 2,4-dichloro-1,1,1,4,4-pentafluorobutane (345lfd) has a boiling point of from about 0° C. to about 130° C., for example, about 0° C. to about 100° C., about 0° C. to about 75° C., about 0° C. to about 50° C., about 0° C. to about 25° C., about 25° C. to about 130° C., about 25° C. to about 100° C., about 25° C. to about 75° C., about 25° C. to about 50° C., about 50° C. to about 130° C., about 50° C. to about 100° C., about 50° C. to about 75° C., about 75° C. to about 130° C., about 75° C. to about 100° C., or about 100° C. to about 130° C., at a pressure of from about 6 psia to about 360 psia, for example, about 6 psia to about 300 psia, about 6 psia to about 250 psia, about 6 psia to about 200 psia, about 6 psia to about 150 psia, about 6 psia to about 100 psia, about 6 psia to about 50 psia, about 50 psia to about 360 psia, about 50 psia to about 300 psia, about 50 psia to about 250 psia, about 50 psia to about 200 psia, about 50 psia to about 150 psia, about 50 psia to about 100 psia, about 100 psia to about 360 psia, about 100 psia to about 300 psia, about 100 psia to about 250 psia, about 100 psia to about 200 psia, about 100 psia to about 150 psia, about 150 psia to about 360 psia, about 150 psia to about 300 psia, about 150 psia to about 250 psia, about 150 psia to about 200 psia, about 200 psia to about 360 psia, about 200 psia to about 300 psia, about 200 psia to about 250 psia, about 250 psia to about 360 psia, about 250 psia to about 300 psia, or about 300 psia to about 360 psia.

In some embodiments, the composition comprises hydrogen fluoride and 2,4-dichloro-1,1,1,4,4-pentafluorobutane (345lfd), wherein the 2,4-dichloro-1,1,1,4,4-pentafluorobutane (345lfd) is present in the composition in an amount effective to form an azeotrope composition with the hydrogen fluoride.

In some embodiments, the composition comprises hydrogen fluoride and 2,4-dichloro-1,1,1,4,4-pentafluorobutane (345lfd), wherein the 2,4-dichloro-1,1,1,4,4-pentafluorobutane (345lfd) is present in the composition in an amount effective to form a homogeneous azeotrope composition with the hydrogen fluoride.

In some embodiments, the composition comprises hydrogen fluoride and 2,4-dichloro-1,1,1,4,4-pentafluorobutane (345lfd), wherein the 2,4-dichloro-1,1,1,4,4-pentafluorobutane (345lfd) is present in the composition in an amount effective to form a heterogeneous azeotrope composition with the hydrogen fluoride.

In some embodiments, the composition comprises hydrogen fluoride and 3-chloro-1,1,4,4,4-pentafluorobut-1-ene (1335czd), wherein the 3-chloro-1,1,4,4,4-pentafluorobut-1-ene (1335czd) is present in the composition in an amount effective to form an azeotrope composition or azeotrope-like composition with the hydrogen fluoride.

In some embodiments, the composition comprising hydrogen fluoride and 3-chloro-1,1,4,4,4-pentafluorobut-1-ene (1335czd) comprises from about 94 to about 75 mole percent hydrogen fluoride, for example, about 94 to about 80, about 94 to about 85, about 94 to about 90, about 90 to about 75, about 90 to about 80, about 90 to about 85, about 85 to about 75, about 85 to about 80, or about 80 to about 75 mole percent hydrogen fluoride.

In some embodiments, the composition comprising hydrogen fluoride and 3-chloro-1,1,4,4,4-pentafluorobut-1-ene (1335czd) comprises about 6 to about 25 mole percent 3-chloro-1,1,4,4,4-pentafluorobut-1-ene (1335czd), for example, about 6 to about 20, about 6 to about 15, about 6 to about 10, about 10 to about 25, about 10 to about 20, about 10 to about 15, about 15 to about 25, about 15 to about 20, or about 20 to about 25 mole percent 3-chloro-1,1,4,4,4-pentafluorobut-1-ene (1335czd).

In some embodiments, the composition comprising hydrogen fluoride and 3-chloro-1,1,4,4,4-pentafluorobut-1-ene (1335czd) comprises from about 94 to about 75 mole percent hydrogen fluoride and from about 6 to about 25 mole percent 3-chloro-1,1,4,4,4-pentafluorobut-1-ene (1335czd).

In some embodiments, the composition comprising hydrogen fluoride and 3-chloro-1,1,4,4,4-pentafluorobut-1-ene (1335czd) has a boiling point of from about 0° C. to about 120° C., for example, about 0° C. to about 100° C., about 0° C. to about 75° C., about 0° C. to about 50° C., about 0° C. to about 25° C., about 25° C. to about 120° C., about 25° C. to about 100° C., about 25° C. to about 75° C., about 25° C. to about 50° C., about 50° C. to about 120° C., about 50° C. to about 100° C., about 50° C. to about 75° C., about 75° C. to about 120° C., about 75° C. to about 100° C., or about 100° C. to about 120° C., at a pressure of from about 7 psia to about 299 psia, for example, about 7 psia to about 250 psia, about 7 psia to about 200 psia, about 7 psia to about 150 psia, about 7 psia to about 100 psia, about 7 psia to about 50 psia, about 50 psia to about 299 psia, about 50 psia to about 250 psia, about 50 psia to about 200 psia, about 50 psia to about 150 psia, about 50 psia to about 100 psia, about 100 psia to about 299 psia, about 100 psia to about 250 psia, about 100 psia to about 200 psia, about 100 psia to about 150 psia, about 150 psia to about 299 psia, about 150 psia to about 250 psia, about 150 psia to about 200 psia, about 200 psia to about 299 psia, about 200 psia to about 250 psia, or about 250 psia to about 299 psia.

In some embodiments, the composition comprises hydrogen fluoride and 3-chloro-1,1,4,4,4-pentafluorobut-1-ene (1335czd), wherein the 3-chloro-1,1,4,4,4-pentafluorobut-1-ene (1335czd) is present in the composition in an amount effective to form an azeotrope composition with the hydrogen fluoride.

In some embodiments, the composition comprises hydrogen fluoride and 3-chloro-1,1,4,4,4-pentafluorobut-1-ene (1335czd), wherein the 3-chloro-1,1,4,4,4-pentafluorobut-1-ene (1335czd) is present in the composition in an amount effective to form a homogeneous azeotrope composition with the hydrogen fluoride.

In some embodiments, the composition comprises hydrogen fluoride and 3-chloro-1,1,4,4,4-pentafluorobut-1-ene (1335czd), wherein the 3-chloro-1,1,4,4,4-pentafluorobut-1-ene (1335czd) is present in the composition in an amount effective to form a heterogeneous azeotrope composition with the hydrogen fluoride.

These compositions may therefore be useful for isolating the azeotropic mixture at a lower temperature compared to the individual components.

The formation of azeotropic or azeotrope-like compositions comprising hydrogen fluoride and 2,4-dichloro-1,1,1,4,4-pentafluorobutane (345lfd) and hydrogen fluoride and 3-chloro-1,1,4,4,4-pentafluorobut-1-ene (1335czd) as described herein allow said compounds to be removed more easily and at lower temperatures from other by-products formed in the synthesis of (E)-1,1,1,4,4,4-hexafluoro-2-butene, allowing for improved purification of the desired products at lower energy and reduced cost. Additionally, said azeotropic compositions can be used in azeotropic distillations to remove hydrogen fluoride from higher boiling compounds that also form azeotropes with hydrogen fluoride.

The present invention further provides a process to remove 2,4-dichloro-1,1,1,4,4-pentafluorobutane (345lfd) from a composition comprising (E)-1,1,1,4,4,4-hexafluoro-2-butene and 2,4-dichloro-1,1,1,4,4-pentafluorobutane, wherein the process comprises forming an azeotropic or azeotrope-like composition of 2,4-dichloro-1,1,1,4,4-pentafluorobutane and HF and removing the azeotrope or azeotrope-like composition from the composition comprising (E)-1,1,1,4,4,4-hexafluoro-2-butene and 2,4-dichloro-1,1,1,4,4-pentafluorobutane (345lfd). The azeotropic or azeotrope-like composition of 2,4-dichloro-1,1,1,4,4-pentafluorobutane and HF comprises from about 83 to about 99.99 mole percent hydrogen fluoride and from about 0.01 to about 17 mole percent 2,4-dichloro-1,1,1,4,4-pentafluorobutane (345lfd).

A process to remove 3-chloro-1,1,4,4,4-pentafluorobut-1-ene (1335czd) from a composition comprising (E)-1,1,1,4,4,4-hexafluoro-2-butene and 3-chloro-1,1,4,4,4-pentafluorobut-1-ene (1335czd), wherein the process comprises forming an azeotropic or azeotrope-like composition of 3-chloro-1,1,4,4,4-pentafluorobut-1-ene (1335czd) and HF and removing the azeotrope or azeotrope-like composition from the composition comprising (E)-1,1,1,4,4,4-hexafluoro-2-butene and 3-chloro-1,1,4,4,4-pentafluorobut-1-ene (1335czd). The azeotropic or azeotrope-like composition of 3-chloro-1,1,4,4,4-pentafluorobut-1-ene (1335czd) and HF comprises from about comprises from about 94 to about 75 mole percent hydrogen fluoride and from about 6 to about 25 mole percent 3-chloro-1,1,4,4,4-pentafluorobut-1-ene (1335czd).

As described herein, 2,4-dichloro-1,1,1,4,4-pentafluorobutane (345lfd) and 3-chloro-1,1,4,4,4-pentafluorobut-1-ene (1335czd) each form azeotropes with HF. Because of these azeotropes, HF may be separated from each of these individual compounds using azeotropic distillation. In addition, the formation of the azeotropes allows separation of other components from reaction mixtures comprising HF and 2,4-dichloro-1,1,1,4,4-pentafluorobutane (345lfd) or 3-chloro-1,1,4,4,4-pentafluorobut-1-ene (1335czd).

The invention will be described in greater detail by way of specific examples. The following examples are offered for illustrative purposes, and are not intended to limit the invention in any manner. Those of skill in the art will readily recognize a variety of non-critical parameters which can be changed or modified to yield essentially the same results.

### EXAMPLES

### Example 1. Vapor-Liquid Equilibrium Analysis

The PTx method is a known method for experimentally measuring vapor-liquid phase equilibrium (VLE) data of a mixture. The measurements can be made either isothermally or isobarically. The isothermal method requires measurement of the total pressure of mixtures of known composition at constant temperature. In this method, the total absolute pressure in a cell of known volume is measured at a constant temperature for various known compositions of the two compounds. The isobaric method requires measurement of the temperature of mixtures of known composition at constant pressure. In this method, the temperature in a cell of known volume is measured at a constant pressure for various known compositions of the two compounds. Use of the PTx Method is described in detail in “Phase Equilibrium in Process Design”, Wiley-Interscience Publisher, 1970, written by Harold R. Null, on pages 124 to 126, the disclosure of which is incorporated herein by reference in its entirety.

The measured data points can be converted into equilibrium vapor and liquid compositions in the PTx cell by using an activity coefficient equation model, such as the Non-Random, Two-Liquid (NRTL) equation, to represent liquid phase nonidealities. Use of an activity coefficient equation, such as the NRTL equation is described in detail in “The Properties of Gases and Liquids,” 4th edition, published by McGraw Hill, written by Reid, Prausnitz and Poling, on pages 241 to 387, and in “Phase Equilibria in Chemical Engineering,” published by Butterworth Publishers, 1985, written by Stanley M. Walas, pages 165 to 244, the disclosure of which is incorporated herein by reference in its entirety. Without wishing to be bound by any theory or explanation, it is believed that the NRTL equation, together with the PTx cell data, sufficiently predicts the vapor-liquid phase equilibrium behavior of the various mixture compositions of the present invention and as well as the behavior of these mixtures in multi-stage separation equipment such as distillation columns.

Since, in both homogeneous and heterogeneous azeotropes, the compositions of the equilibrium vapor and liquid phases (overall liquid phase composition for heterogeneous azeotropes) are equal, azeotropic compositions can be determined over a range of temperatures or pressures by: (1) measuring vapor-liquid equilibrium data over the full composition range at preferably two or more temperatures or pressures, (2) fitting the adjustable parameters of a vapor-liquid equilibrium model such as the Peng-Robinson equation of state and/or the NRTL equation to the experimental data, and (3) using the resulting model parameters to calculate vapor-liquid phase equilibrium and determine the point at which the vapor and liquid phase compositions are equal. This method was used to generate Tables 1-2 and the azeotropic compositions described throughout the Examples. For mixtures that form heterogeneous azeotropes, unless stated otherwise, the reported liquid-phase compositions are those of the overall liquid phase.

### Example 2. Azeotropic Compositions of Hydrogen Fluoride and 2,4-Dichloro-1,1,1,4,4-Pentafluorobutane (345lfd)

The azeotropic ranges for mixtures of hydrogen fluoride and 2,4-dichloro-1,1,1,4,4-pentafluorobutane (345lfd) from 0° C. to 130° C. are summarized in Table 1.

TABLE 1

| | | Vapor-Phase | | Liquid 1 | | Liquid 2 | |
|---|---|---|---|---|---|---|---|
| Azeotropic | Azeotropic | Composition | | Composition | | Composition | |
| Temperature | Pressure | (Mole Percent) | | (Mole Percent) | | (Mole Percent) | |
| (° C.) | (psia) | HF | 345lfd | HF | 345lfd | HF | 345lfd |
| 0 | 6.99 | 99.99 | 0.01 | 99.99 | 0.01 | | |
| 10 | 10.43 | 98.53 | 1.47 | 98.53 | 1.47 | | |
| 20 | 15.24 | 97.41 | 2.59 | 97.41 | 2.59 | | |
| 30 | 21.80 | 96.64 | 3.36 | 96.64 | 3.36 | | |
| 40 | 30.56 | 95.88 | 4.12 | 96.59 | 3.41 | 7.91 | 92.09 |
| 50 | 42.08 | 95.07 | 4.93 | 96.19 | 3.82 | 9.81 | 90.19 |
| 60 | 57.04 | 94.15 | 5.85 | 95.72 | 4.28 | 12.03 | 87.97 |
| 70 | 76.25 | 93.12 | 6.88 | 95.17 | 4.83 | 14.61 | 85.39 |
| 80 | 100.72 | 91.95 | 8.05 | 94.53 | 5.47 | 17.56 | 82.44 |
| 90 | 131.69 | 90.65 | 9.35 | 93.78 | 6.22 | 20.92 | 79.08 |
| 100 | 170.84 | 89.16 | 10.84 | 92.88 | 7.12 | 24.70 | 75.30 |
| 110 | 220.62 | 87.38 | 12.62 | 91.79 | 8.21 | 28.94 | 71.06 |
| 120 | 285.54 | 84.92 | 15.08 | 90.44 | 9.56 | 33.69 | 66.31 |
| 130 | 359.23 | 83.24 | 16.76 | 88.73 | 11.27 | 39.03 | 60.97 |

### Example 3. Azeotropic Compositions of Hydrogen Fluoride and 3-Chloro-1,1,4,4,4-Pentafluorobut-1-ene (1335czd)

The azeotropic ranges for mixtures of hydrogen fluoride and 3-chloro-1,1,4,4,4-pentafluorobut-1-ene (1335czd) from 0° C. to 120° C. are summarized in Table 2.

TABLE 2

| | | Vapor-Phase | | Liquid | |
|---|---|---|---|---|---|
| Azeotropic | Azeotropic | Composition | | Composition | |
| Temperature | Pressure | (Mole Percent) | | (Mole Percent) | |
| (° C.) | (psia) | HF | 1335czd | HF | 1335czd |
| 0 | 7.64 | 93.22 | 6.78 | 93.20 | 6.80 |
| 10 | 11.55 | 92.00 | 8.00 | 92.13 | 7.87 |
| 20 | 16.98 | 90.59 | 9.41 | 90.53 | 9.47 |
| 30 | 24.36 | 89.20 | 10.80 | 89.20 | 10.80 |
| 40 | 34.20 | 87.76 | 12.24 | 87.73 | 12.27 |
| 50 | 47.06 | 86.30 | 13.70 | 86.27 | 13.73 |
| 60 | 63.61 | 84.86 | 15.14 | 84.80 | 15.20 |
| 70 | 84.62 | 83.48 | 16.52 | 83.47 | 16.53 |
| 80 | 110.98 | 82.12 | 17.88 | 82.13 | 17.87 |
| 90 | 143.81 | 80.78 | 19.22 | 80.80 | 19.20 |
| 100 | 184.50 | 79.39 | 20.61 | 79.33 | 20.67 |
| 110 | 235.03 | 77.89 | 22.11 | 77.87 | 22.13 |
| 120 | 298.75 | 75.74 | 24.26 | 75.47 | 24.53 |

### Embodiments

In an embodiment 1, the present application provides a composition, comprising: i) hydrogen fluoride; and ii) a compound selected from 2,4-dichloro-1,1,1,4,4-pentafluorobutane (345lfd) and 3-chloro-1,1,4,4,4-pentafluorobut-1-ene (1335czd); wherein the 2,4-dichloro-1,1,1,4,4-pentafluorobutane (345lfd) or 3-chloro-1,1,4,4,4-pentafluorobut-1-ene (1335czd) is present in the composition in an amount effective to form an azeotrope composition or azeotrope-like composition with the hydrogen fluoride.

In an embodiment 2, the composition of the embodiment 1 comprises hydrogen fluoride and 2,4-dichloro-1,1,1,4,4-pentafluorobutane (345lfd), wherein the 2,4-dichloro-1,1,1,4,4-pentafluorobutane (345lfd) is present in the composition in an amount effective to form an azeotrope composition or azeotrope-like composition with the hydrogen fluoride.

In an embodiment 3, the composition of the embodiment 2 comprises from about 83 to about 99.99 mole percent hydrogen fluoride and from about 0.01 to about 17 mole percent 2,4-dichloro-1,1,1,4,4-pentafluorobutane (345lfd).

In an embodiment 4, the composition of the embodiment 2 comprises from about 83 to about 99 mole percent hydrogen fluoride and from about 1 to about 17 mole percent 2,4-dichloro-1,1,1,4,4-pentafluorobutane (345lfd).

In an embodiment 5, the composition of the embodiment 2 comprises from about 83 to about 95 mole percent hydrogen fluoride and from about 5 to about 17 mole percent 2,4-dichloro-1,1,1,4,4-pentafluorobutane (345lfd),

In an embodiment 6, the composition of the embodiment 2 comprises from about 83 to about 90 mole percent hydrogen fluoride and from about 10 to about 17 mole percent 2,4-dichloro-1,1,1,4,4-pentafluorobutane (345lfd),

In an embodiment 7, the composition of the embodiment 2 comprises from about 83 to about 85 mole percent hydrogen fluoride and from about 15 to about 17 mole percent 2,4-dichloro-1,1,1,4,4-pentafluorobutane (345lfd),

In an embodiment 8, the composition of the embodiment 2 comprises from about 85 to about 99.99 mole percent hydrogen fluoride and from about 0.01 to about 15 mole percent 2,4-dichloro-1,1,1,4,4-pentafluorobutane (345lfd),

In an embodiment 9, the composition of the embodiment 2 comprises from about 85 to about 99 mole percent hydrogen fluoride and from about 0.01 to about 15 mole percent 2,4-dichloro-1,1,1,4,4-pentafluorobutane (345lfd),

In an embodiment 10, the composition of the embodiment 2 comprises from about 85 to about 95 mole percent hydrogen fluoride and from about 0.01 to about 15 mole percent 2,4-dichloro-1,1,1,4,4-pentafluorobutane (345lfd),

In an embodiment 11, the composition of the embodiment 2 comprises from about 85 to about 90 mole percent hydrogen fluoride and from about 0.01 to about 15 mole percent 2,4-dichloro-1,1,1,4,4-pentafluorobutane (345lfd),

In an embodiment 12, the composition of the embodiment 2 comprises from about 90 to about 99.99 mole percent hydrogen fluoride and from about 0.01 to about 10 mole percent 2,4-dichloro-1,1,1,4,4-pentafluorobutane (345lfd),

In an embodiment 13, the composition of the embodiment 2 comprises from about 90 to about 99 mole percent hydrogen fluoride and from about 1 to about 10 mole percent 2,4-dichloro-1,1,1,4,4-pentafluorobutane (345lfd),

In an embodiment 14, the composition of the embodiment 2 comprises from about 90 to about 95 mole percent hydrogen fluoride and from about 5 to about 10 mole percent 2,4-dichloro-1,1,1,4,4-pentafluorobutane (345lfd),

In an embodiment 15, the composition of the embodiment 2 comprises from about 95 to about 99.99 mole percent hydrogen fluoride and from about 0.01 to about 5 mole percent 2,4-dichloro-1,1,1,4,4-pentafluorobutane (345lfd),

In an embodiment 16, the composition of the embodiment 2 comprises from about 95 to about 99 mole percent hydrogen fluoride and from about 1 to about 5 mole percent 2,4-dichloro-1,1,1,4,4-pentafluorobutane (345lfd),

In an embodiment 17, the composition of the embodiment 2 comprises from about 99 to about 99.99 mole percent hydrogen fluoride and from about 0.01 to about 1 mole percent 2,4-dichloro-1,1,1,4,4-pentafluorobutane (345lfd).

In an embodiment 18, the composition of the embodiment 2 has a boiling point of from about 0° C. to about 130° C. at a pressure of from about 6 psia to about 360 psia.

In an embodiment 19, the composition of the embodiment 2 has a boiling point of from about 0° C. to about 130° C. at a pressure of from about 6 psia to about 360 psia.

In an embodiment 20, the composition of the embodiment 1 comprises hydrogen fluoride and 3-chloro-1,1,4,4,4-pentafluorobut-1-ene (1335czd), wherein the 3-chloro-1,1,4,4,4-pentafluorobut-1-ene (1335czd) is present in the composition in an amount effective to form an azeotrope composition or azeotrope-like composition with the hydrogen fluoride.

In an embodiment 21, the composition of the embodiment 20 comprises from about 94 to about 75 mole percent hydrogen fluoride and from about 6 to about 25 mole percent 3-chloro-1,1,4,4,4-pentafluorobut-1-ene (1335czd).

In an embodiment 22, the composition of the embodiment 21 comprises from about 94 to about 80 mole percent hydrogen fluoride and from about 6 to about 20 mole percent 3-chloro-1,1,4,4,4-pentafluorobut-1-ene (1335czd).

In an embodiment 23, the composition of the embodiment 21 comprises from about 94 to about 85 mole percent hydrogen fluoride and from about 6 to about 15 mole percent 3-chloro-1,1,4,4,4-pentafluorobut-1-ene (1335czd).

In an embodiment 24, the composition of the embodiment 21 comprises from about 94 to about 90 mole percent hydrogen fluoride and from about 6 to about 10 mole percent 3-chloro-1,1,4,4,4-pentafluorobut-1-ene (1335czd).

In an embodiment 25, the composition of the embodiment 21 comprises from about 90 to about 75 mole percent hydrogen fluoride and from about 10 to about 25 mole percent 3-chloro-1,1,4,4,4-pentafluorobut-1-ene (1335czd).

In an embodiment 26, the composition of the embodiment 21 comprises from about 90 to about 80 mole percent hydrogen fluoride and from about 10 to about 20 mole percent 3-chloro-1,1,4,4,4-pentafluorobut-1-ene (1335czd).

In an embodiment 27, the composition of the embodiment 21 comprises from about 90 to about 85 mole percent hydrogen fluoride and from about 10 to about 15 mole percent 3-chloro-1,1,4,4,4-pentafluorobut-1-ene (1335czd).

In an embodiment 28, the composition of the embodiment 21 comprises from about 85 to about 75 mole percent hydrogen fluoride and from about 15 to about 25 mole percent 3-chloro-1,1,4,4,4-pentafluorobut-1-ene (1335czd).

In an embodiment 29, the composition of the embodiment 21 comprises from about 85 to about 80 mole percent hydrogen fluoride and from about 15 to about 20 mole percent 3-chloro-1,1,4,4,4-pentafluorobut-1-ene (1335czd).

In an embodiment 30, the composition of the embodiment 21 comprises from about 80 to about 75 mole percent hydrogen fluoride and from about 20 to about 25 mole percent 3-chloro-1,1,4,4,4-pentafluorobut-1-ene (1335czd).

In an embodiment 31, the composition of the embodiment 21 has a boiling point of from about 0° C. to about 120° at a pressure of from about 7 psia to about 299 psia.

In an embodiment 32, the composition of any of the embodiments 1 to 31 is a homogeneous azeotrope or homogeneous azeotrope-like composition.

In an embodiment 33, the composition of any of the embodiments 1 to 31 is a is a homogeneous azeotrope.

In an embodiment 34, the composition of any of the embodiments 1 to 31 is a heterogeneous azeotrope or heterogeneous azeotrope-like composition.

In an embodiment 35, the composition of any of the embodiments 1 to 31 is a heterogeneous azeotrope.

In an embodiment 36, the composition of any of the embodiments 1 to 31 is an azeotrope-like composition.

In an embodiment 37, the present application provides a process to remove 2,4-dichloro-1,1,1,4,4-pentafluorobutane (345lfd) from a composition comprising (E)-1,1,1,4,4,4-hexafluoro-2-butene and 2,4-dichloro-1,1,1,4,4-pentafluorobutane, wherein the process comprises forming an azeotropic or azeotrope-like composition of 2,4-dichloro-1,1,1,4,4-pentafluorobutane and HF, and removing the azeotrope or

1. A composition comprising:
i) hydrogen fluoride; and
ii) a compound selected from 2,4-dichloro-1,1,1,4,4-pentafluorobutane (345lfd) and 3-chloro-1,1,4,4,4-pentafluorobut-1-ene (1335czd);
wherein the 2,4-dichloro-1,1,1,4,4-pentafluorobutane (345lfd) or 3-chloro-1,1,4,4,4-pentafluorobut-1-ene (1335czd) is present in the composition in an amount effective to form an azeotrope composition or azeotrope-like composition with the hydrogen fluoride.

2. The composition of claim 1, wherein the composition comprises hydrogen fluoride and 2,4-dichloro-1,1,1,4,4-pentafluorobutane (345lfd), wherein the composition comprises from about 83 to about 99.99 mole percent hydrogen fluoride and from about 0.01 to about 17 mole percent 2,4-dichloro-1,1,1,4,4-pentafluorobutane (345lfd).

3. (canceled)

4. The composition of claim 2, wherein the composition comprises from about 83 to about 99.99 mole percent hydrogen fluoride; or from about 83 to about 99 mole percent hydrogen fluoride, or from about 83 to about 95 mole percent hydrogen fluoride; or from about 83 to about 90 mole percent hydrogen fluoride; or from about 83 to about 85 mole percent hydrogen fluoride; or from about 85 to about 99.99 mole percent hydrogen fluoride; or from about 85 to about 99 mole percent hydrogen fluoride; or from about 85 to about 95 mole percent hydrogen fluoride; or from about 85 to about 90 mole percent hydrogen fluoride; or from about 90 to about 99.99 mole percent hydrogen fluoride; or from about 90 to about 99 mole percent hydrogen fluoride; or from about 90 to about 95 mole percent hydrogen fluoride; or from about 95 to about 99.99 mole percent hydrogen fluoride; or from about 95 to about 99 mole percent hydrogen fluoride; or from about 99 to about 99.99 mole percent hydrogen fluoride.

5. The composition of claim 2, wherein the composition comprises from about 0.01 to about 17 mole percent 2,4-dichloro-1,1,1,4,4-pentafluorobutane (345lfd); or from about 0.01 to about 15 mole percent 2,4-dichloro-1,1,1,4,4-pentafluorobutane (345lfd); or from about 0.01 to about 10 mole percent 2,4-dichloro-1,1,1,4,4-pentafluorobutane (345lfd); or from about 0.01 to about 5 mole percent 2,4-dichloro-1,1,1,4,4-pentafluorobutane (345lfd); or from about 0.01 to about 1 mole percent 2,4-dichloro-1,1,1,4,4-pentafluorobutane (345lfd); or from about 1 to about 17 mole percent 2,4-dichloro-1,1,1,4,4-pentafluorobutane (345lfd); or from about 1 to about 15 mole percent 2,4-dichloro-1,1,1,4,4-pentafluorobutane (345lfd); or from about 1 to about 10 mole percent 2,4-dichloro-1,1,1,4,4-pentafluorobutane (345lfd); or from about 1 to about 5 mole percent 2,4-dichloro-1,1,1,4,4-pentafluorobutane (345lfd); or from about 5 to about 17 mole percent 2,4-dichloro-1,1,1,4,4-pentafluorobutane (345lfd); or from about 5 to about 15 mole percent 2,4-dichloro-1,1,1,4,4-pentafluorobutane (345lfd); or from about 5 to about 10 mole percent 2,4-dichloro-1,1,1,4,4-pentafluorobutane (345lfd); or from about 10 to about 17 mole percent 2,4-dichloro-1,1,1,4,4-pentafluorobutane (345lfd); or from about 10 to about 15 mole percent 2,4-dichloro-1,1,1,4,4-pentafluorobutane (345lfd); or from about 15 to about 17 mole percent 2,4-dichloro-1,1,1,4,4-pentafluorobutane (345lfd).

6. The composition of claim 2, wherein the composition comprises from about 88.7 to about 99.99 mole percent hydrogen fluoride and from about 0.01 to about 11.3 mole percent 2,4-dichloro-1,1,1,4,4-pentafluorobutane (345lfd).

7. The composition of claim 2, wherein the composition has a boiling point of from about 0° C. to about 130° C. at a pressure of from about 6 psia to about 360 psia.

8. The composition of claim 2, wherein the 2,4-dichloro-1,1,1,4,4-pentafluorobutane (345lfd) is present in the composition in an amount effective to form an azeotrope composition with the hydrogen fluoride.

9. The composition of claim 8, wherein the 2,4-dichloro-1,1,1,4,4-pentafluorobutane (345lfd) is present in the composition in an amount effective to form a homogeneous azeotrope composition with the hydrogen fluoride.

10. The composition of claim 8, wherein the 2,4-dichloro-1,1,1,4,4-pentafluorobutane (345lfd) is present in the composition in an amount effective to form a heterogeneous azeotrope composition with the hydrogen fluoride.

11. The composition of claim 2, wherein the 2,4-dichloro-1,1,1,4,4-pentafluorobutane (345lfd) is present in the composition in an amount effective to form an azeotrope-like composition with the hydrogen fluoride.

12. The composition of claim 1, wherein the composition comprises hydrogen fluoride and 3-chloro-1,1,4,4,4-pentafluorobut-1-ene (1335czd), wherein the composition comprises from about 94 to about 75 mole percent hydrogen fluoride and from about 6 to about 25 mole percent 3-chloro-1,1,4,4,4-pentafluorobut-1-ene (1335czd).

13. (canceled)

14. The composition of claim 12, wherein the composition comprises from about 94 to about 80 mole percent hydrogen fluoride, or about 94 to about 85 mole percent hydrogen fluoride, or about 94 to about 90 mole percent hydrogen fluoride, or about 90 to about 75 mole percent hydrogen fluoride, or about 90 to about 80 mole percent hydrogen fluoride, or about 90 to about 85 mole percent hydrogen fluoride, or about 85 to about 75 mole percent hydrogen fluoride, or about 85 to about 80 mole percent hydrogen fluoride, or about 80 to about 75 mole percent hydrogen fluoride.

15. The composition of claim 12, wherein the composition comprises for about 6 to about 20 mole percent 3-chloro-1,1,4,4,4-pentafluorobut-1-ene (1335czd), or about 6 to about 15 mole percent 3-chloro-1,1,4,4,4-pentafluorobut-1-ene (1335czd), or about 6 to about 10 mole percent 3-chloro-1,1,4,4,4-pentafluorobut-1-ene (1335czd), or about 10 to about 25 mole percent 3-chloro-1,1,4,4,4-pentafluorobut-1-ene (1335czd), or about 10 to about 20 mole percent 3-chloro-1,1,4,4,4-pentafluorobut-1-ene (1335czd), or about 10 to about 15 mole percent 3-chloro-1,1,4,4,4-pentafluorobut-1-ene (1335czd), or about 15 to about 25 mole percent 3-chloro-1,1,4,4,4-pentafluorobut-1-ene (1335czd), or about 15 to about 20 mole percent 3-chloro-1,1,4,4,4-pentafluorobut-1-ene (1335czd), or about 20 to about 25 mole percent 3-chloro-1,1,4,4,4-pentafluorobut-1-ene (1335czd).

16. The composition of claim 12 wherein the composition has a boiling point of from about 0° C. to about 120° C. at a pressure of from about 7 psia to about 299 psia.

17. The composition of claim 12, wherein the 3-chloro-1,1,4,4,4-pentafluorobut-1-ene (1335czd) is present in the composition in an amount to form an azeotrope composition with hydrogen fluoride.

18. The composition of claim 12, wherein the 3-chloro-1,1,4,4,4-pentafluorobut-1-ene (1335czd) is present in the composition in an amount to form a homogeneous azeotrope composition with hydrogen fluoride.

19. The composition of claim 12, wherein the 3-chloro-1,1,4,4,4-pentafluorobut-1-ene (1335czd) is present in the composition in an amount to form a heterogeneous azeotrope composition with hydrogen fluoride.

20. The composition of claim 12, wherein the 3-chloro-1,1,4,4,4-pentafluorobut-1-ene (1335czd) is present in the composition in an amount to form an azeotrope-like composition with hydrogen fluoride.

21. A process to remove 2,4-dichloro-1,1,1,4,4-pentafluorobutane (345lfd) from a composition comprising (E)-1,1,1,4,4,4-hexafluoro-2-butene and 2,4-dichloro-1,1,1,4,4-pentafluorobutane, comprising forming an azeotropic or azeotrope-like composition of 2,4-dichloro-1,1,1,4,4-pentafluorobutane and HF and removing the azeotrope or azeotrope-like composition from the composition comprising (E)-1,1,1,4,4,4-hexafluoro-2-butene and 2,4-dichloro-1,1,1,4,4-pentafluorobutane (345lfd).

22. The process of claim 21 wherein the azeotropic or azeotrope-like composition of 2,4-dichloro-1,1,1,4,4-pentafluorobutane and HF comprises from about 83 to about 99.99 mole percent hydrogen fluoride and from about 0.01 to about 17 mole percent 2,4-dichloro-1,1,1,4,4-pentafluorobutane (345lfd).

23. A process to remove 3-chloro-1,1,4,4,4-pentafluorobut-1-ene (1335czd) from a composition comprising (E)-1,1,1,4,4,4-hexafluoro-2-butene and 3-chloro-1,1,4,4,4-pentafluorobut-1-ene (1335czd), wherein the process comprises forming an azeotropic or azeotrope-like composition of 3-chloro-1,1,4,4,4-pentafluorobut-1-ene (1335czd) and HF and removing the azeotrope Page 7 or azeotrope-like composition from the composition comprising (E)-1,1,1,4,4,4-hexafluoro-2-butene and 3-chloro-1,1,4,4,4-pentafluorobut-1-ene (1335czd).

24. The process of claim 23 wherein the azeotropic or azeotrope-like composition of 3-chloro-1,1,4,4,4-pentafluorobut-1-ene (1335czd) and HF comprises from about comprises from about 94 to about 75 mole percent hydrogen fluoride and from about 6 to about 25 mole percent 3-chloro-1,1,4,4,4-pentafluorobut-1-ene (1335czd).
